(19) United States
(12) Patent Application Publication
(10) Pub. No.: US 2006/0155386 A1
(43) Pub. Date: Jul. 13, 2006

(54) Electromyographic sensor

(76) Inventors: David L. Wells, Etobicoke (CA); John W. Bishop, Toronto (CA); Paul K. O'Brien, Toronto (CA); Kaveh Seyed Momen, Toronto (CA)

Correspondence Address: TORYS LLP, 79 WELLINGTON ST. WEST, SUITE 3000, TORONTO, ON, M5K 1N2, CA

(21) Appl. No.: 11/030,871

(22) Filed: Jan. 10, 2005

(51) Int. Cl. A61F 2/70; A61B 5/04; A61B 5/103

(52) U.S. Cl. 623/25; 600/546; 600/595

(57) ABSTRACT

An electromyographic sensor is provided. The sensor includes electrodes for receiving signals from tissue when the electrodes are placed in contact with the tissue. The sensor also includes circuitry for converting the signals into a format suitable for transmission. the sensor also includes a transmitter for transmitting the signals to a receiver. The receiver can be part of a controller for a prosthetic limb, or the like.

### FIELD OF THE INVENTION

The present invention relates generally to control of prostheses and the like and more particularly relates to an electromyographic sensor.

### BACKGROUND OF THE INVENTION

Electromyographic (“EMG”) sensors are well known. EMG sensors in particular are known for their use in the control of electrically powered prosthetic systems. An individual can have an EMG sensor affixed to a portion of his or her body, and issue instructions to a prosthesis attached to the EMG sensor by voluntarily sending muscular signals to the EMG sensor. The EMG sensor detects the electric signal of the muscles and generates a control or input signal that is delivered to the prosthetic system. In this manner, the user voluntarily controls the prosthesis. One example of a prior art EMG sensor is the Otto Bock brand of myographic electrode (EMG sensor), from Otto Bock, Two Carlson Parkway North, Suite 100, Minneapolis, Minn. 55447-4467, model number 13E125.

Existing EMG sensors used in the control of electrically powered prosthetic systems, including those found in products from Otto Bock, such as their 12K42 and 12K50 ErgoArm Elbows and 12K44 ErgoArm Elbow Hybrd Plus, all utilize a wiring system that connects the electrode (sensor) to control electronics. Users of prosthetic systems utilizing currently existing EMG sensors frequently encounter problems associated with the wiring system. Examples of problems associated with the wiring system include wire defects and damage, and wire connection errors, which can all be difficult to detect. In addition, existing wiring systems are often mechanically complex due to the complexity of wire routing between the electrode and control electronics. Wiring systems also occupy valuable space, and thereby increase the size of prostheses, add weight and impair agility and increase user fatigue. Even small reductions in weight can have significant performance improvements.

The physical impact and damage from daily usage coupled with the need for sensitive proportional control in prosthetic systems, make high demands on the reliability and stability of control signals from EMG sensors. As such, prosthetic systems utilizing existing EMG sensors are limited by the reliability of their wiring systems.

Telemetry of biological data has been researched for many years (Stoller, 1986; Jeutter, 1982). EMG data has proven itself useful in rehabilitation. It has been used to control myoelectric prostheses for many years and has been shown to be useful for human interfaces and gait analysis, as well (Giuffrida J P and Crago P E, “Reciprocal EMG control of elbow extension by FES,” IEEE Trans Neural Syst Rehabil Eng, 2001, December; 9(4), pp. 338-45; Brudny J, Hammerschlag P E, Cohen N L and Ransohoff J, “Electromyographic rehabilitation of facial function and introduction of a facial paralysis grading scale for hypoglossal-facial nerve anastomosis,” Laryngoscope, 1988, April; 98(4), pp. 405-10; Manal K, Gonzalez R V, Lloyd D G and Buchanan T S, “A real-time EMG-driven virtual arm,” Comput Biol Med, 2002, January; 32(1), pp. 25-36; Barreto A B, Scargle S D and Adjouadi M, “A practical EMG-based human-computer interface for users with motor disabilities,” J Rehabil Res Dev, 2000, January-February; 37(1), pp. 53-63; Chang G C, Kang W J, Luh J J, Cheng C K, Lai J S, Chen J J and Kuo T S, “Real-time implementation of electromyogram pattern recognition as a control command of man-machine interface,” Med Eng Phys, 1996, October; 18(7), pp. 529-37; Quanbury A O, Foley C D, Winter D A, Letts R M, and Steinke T, “Clinical telemetry of EMG and temporal information during gait,” Biotelemetry, 1976; 3(3-4), pp. 129-137; Letts R M, Winter D A, and Quanbury A O, “Locomotion studies as an aid in clinical assessment of childhood gait,” Can Med Assoc J, 1975, May 3; 112(9), pp. 1091-5; Winter D A, “Pathologic gait diagnosis with computer-averaged electromyographic profiles,” Arch Phys Med Rehabil, 1984, July; 65(7), pp. 393-8; Perry J, Bontrager E L, Bogey R A, Gronley J K and Barnes L A, “The Rancho EMG analyzer: a computerized system for gait analysis,” J Biomed Eng, 1993, November; 15(6), pp. 487-96; and Harlaar J, Redmeijer R A, Tump P, Peters R and Hautus E, “The SYBAR system: integrated recording and display of video, EMG, and force plate data,” Behav Res Methods Instrum Comput, 2000, February; 32(1), pp. 11-6). Specifically, wireless transmission of EMG data has been used in research for several years. Previous wireless systems have been large, power consumptive, and unwieldy. Only recently with the advent of new technologies has miniaturization and lowered power consumption been available for wireless EMG systems. Several systems have been developed for research (Mohseni P, Nagarajan K, Ziaie B, Najafi K, and Crary S B, “An ultralight biotelemetry backpack for recording EMG signals in moths,” IEEE Trans Biomed Eng, 2001, June; 48(6), pp. 734-7; Langenbach G E, van Ruijven L J, and van Eijden T M, “A telemetry system to chronically record muscle activity in middle-sized animals,” J Neurosci Methods, 2002, Mar 15; 114(2), pp. 197-203; and Meile T and Zittel T T, “Telemetric small intestinal motility recording in awake rats: a novel approach,” Eur Surg Res, 2002, May-June; 34(3), pp. 271-4). One system, Noraxon TeleMyo 2400T, has recently become available commercially. Such systems have demonstrated the potential for miniaturized wireless EMG transmission and have demonstrated that further development of systems for wireless EMG transmission is desirable. Indeed, a self-contained wireless EMG system addressing the problems of rehabilitation systems such as prosthetics and communication and computer access, has not yet been developed.

### SUMMARY OF THE INVENTION

It is an object of the present invention to provide a novel electromyographic sensor that obviates or mitigates at least one of the above-identified disadvantages of the prior art.

A unique wireless electromyogram (EMG) electrode prototype is provided. It can be used for control of powered, upper-extremity prostheses and for Morse code generation by people with conditions such as Amyotrophic Lateral Sclerosis (ALS), and other conditions that limit accessibility to communications and computer equipment. The electrode uses a standard differential pair of metal contacts and a ground contact at the skin interface. It also uses state-of-the-art electronics for wireless data transmission. The EMG electrode is an improvement over commercially available electrodes because it eliminates the need for a wiring harness to connect the electrode to control electronics. This addresses frustrating problems associated with wiring, especially in prostheses—wire failure and wire routing. The new electrode will improve reliability and decrease the mechanical complexity caused by routing for wiring harnesses. The EMG electrode will also be a means of input for communication and computer access which will not hinder or tether the user since it does not use wires for transmission of signals. The electrode will also be useful for untethered measurement of EMG for use in gait analysis.

The desire to use wireless technology for transmitting sensor data has been around for a long time; however, the technology to create systems at the size needed and at a low cost was not available. The technology is now available. Developments in the cellular communications industry and exercise monitoring industry have created the technology infrastructure necessary to make these systems practical and reliable.

An aspect of the invention provides an electromyographic sensor comprising electrodes for placement in contact with tissue. The electrodes are for receiving electrical signals from the tissue. The sensor also includes a circuit connected to the electrodes for converting the signals into a format suitable for wireless transmission. The sensor also includes a transmitter connected to the circuit and for broadcasting the signals.

The circuit can be based on at least one of analog signal processing; digital signal processing; and adaptive filtering.

The sensor can further comprise a receiver. The receiver is operable to receive additional signals that include instructions for instructing how the circuit is to process the signals.

The broadcasting of the signal can be based on radio frequency, infra-red and/or acoustic technology, or other wireless formats.

The broadcasting can be based on at least one of amplitude modulated analog signals; frequency modulated analog signals; code division multiple access digital signals and orthogonal frequency multiple access digital signals.

The circuit can be further operable to add an identifier to the signal such that the sensor is uniquely identifiable.

The transmitter can include means for varying transmission power thereof according to a desired operating range.

Power for the circuit can be provided by a battery housed within the sensor, such as a rechargeable battery based on NiMH or other battery chemistries such as Lithium-ion (“Li-ion). The battery can be configured to be rechargeable via wireless means.

Another aspect of the invention provides a man-machine interface based on an electromyographic sensor of the above-mentioned type. The man machine interface can be selected from a group consisting of a pointing device such as a computer mouse, a trackball, a tablet and others; a sensor for a prosthesis; a sensor for a rehabilitation device; a sensor for gait or movement analysis. These interfaces can be used, for example, to optimize exercise and training, to evaluate workplaces, and to improve ergonomics.

Another aspect of the invention provides a prosthetic system comprising an electromechanical prosthetic limb and a controller connected to the limb for issuing movement instructions thereto. The controller includes a wireless receiver. The system also includes an electromyographic sensor of the above-mentioned type.

Another aspect of the invention provides a movement analysis system comprising a plurality of electromyographic sensors of the above-mentioned type and a computing apparatus having a receiver operable to receive the signals, the computing apparatus operable to generate a computerized representation of the movement based on the signals.

Another aspect of the invention provides an electromyography method comprising the steps of:
receiving electrical signals from electrodes in contact with tissue;
converting the signals into a format suitable for wireless transmission; and,
wirelessly broadcasting the signals.

### BRIEF DESCRIPTION OF THE DRAWINGS

The invention will now be described by way of example only, and with reference to the accompanying drawings, in which:

FIG. 1 is a representation of a prior art prosthetic system including a prior art electromyographic sensor;

FIG. 2 is a representation of a prosthetic system including an electromyographic sensor in accordance with an embodiment of the invention;

FIG. 3 is a block diagram of the sensor in FIG. 2;

FIG. 4 is a block diagram of the transceiver in FIG. 2;

FIG. 5 is a left side view of the sensor in FIG. 2;

FIG. 6 is a bottom view of the sensor in FIG. 2;

FIG. 7 is a front view of the sensor in FIG. 2; and,

FIG. 8 is an exploded front view of the sensor of FIG. 2.

### DETAILED DESCRIPTION OF THE INVENTION

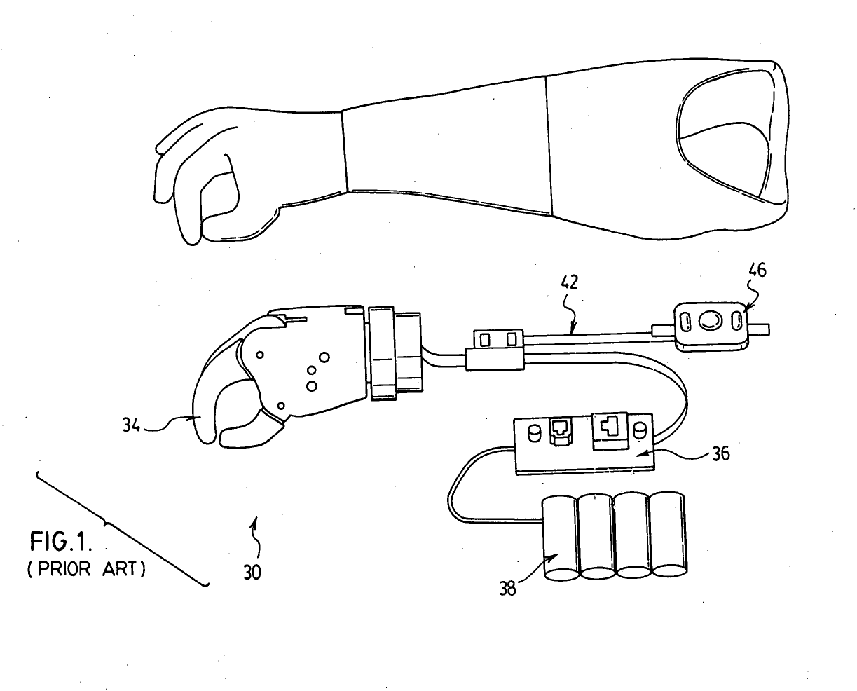

Referring now to FIG. 1, a prior art prosthetic system is indicated generally at 30. System 30 includes an electromechanical prosthetic limb 34 that is connected to a controller 36 having a separate power supply 38. Controller 36 is connected via a ribbon cable 42 to an electromyographic sensor 46. Sensor 46 is an Otto Bock brand of myographic electrode, model number 13E125. Sensor 46 can be affixed to any tissue on the wearer of limb 34 that can be activated by the wearer so that impulses can be sent to sensor 46 for the purposes of controlling limb 34. Ribbon cable 42 carries power to sensor 46 from power supply 38. Cable 42 also carries signals generated by sensor 46 to controller 36. In turn, controller 36 is operable to interpret such received signals and issue instructions to limb 34 to cause limb 34 to move in a particular fashion. Cable 42 presents certain problems for system 30, in that its length can limit the tissue that can be used by the wearer. As yet a further problem, cable 42 can become tangled and therefore interfere with the overall operation of limb 34. Still further problems can arise, such as wire breakage and the presence of the cable adds overall mass to system 30.

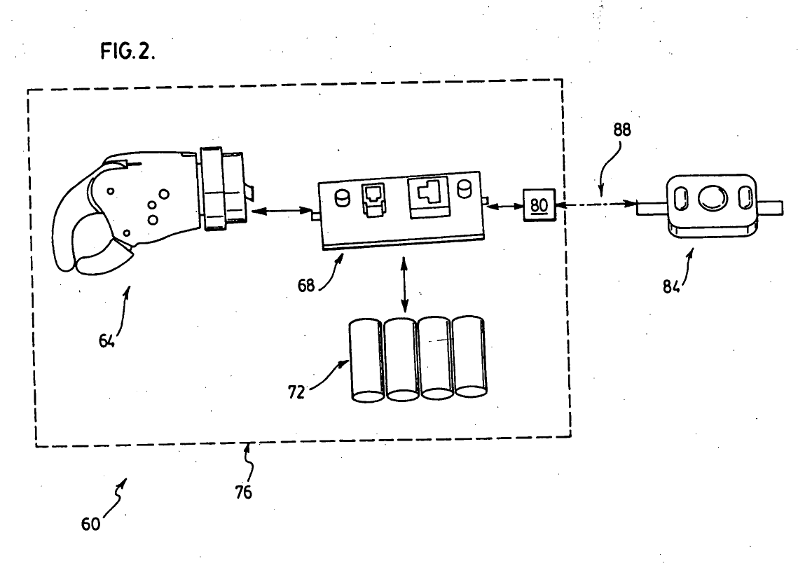

Referring now to FIG. 2, a prosthetic system in accordance with an embodiment of the invention is indicated generally at 60. System 60 comprises an electromechanical prosthetic limb 64 that is connected to a controller 68 having a separate power supply 72. Collectively, limb 64, controller 68 and power supply 72 can be viewed as a man machine interface 76, and other types of man machine interfaces within the scope of the invention will be discussed below.

System 60 also includes a wireless transceiver 80 that connects to controller 68. System 60 also includes a wireless electromyographic sensor 84 that is operable to communicate with controller 68 via transceiver 80 over a wireless link 88.

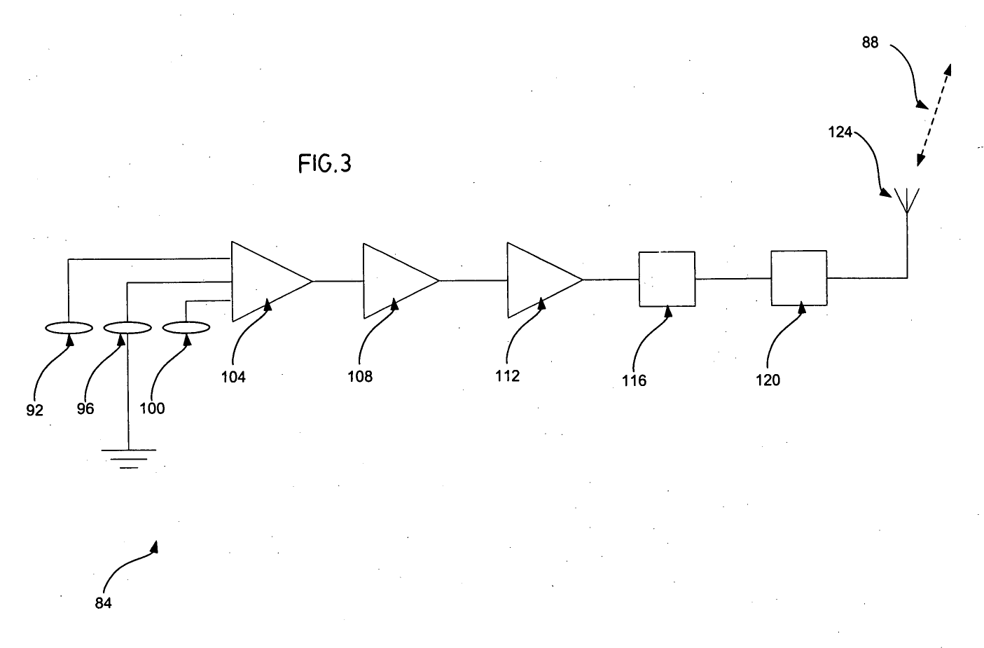

Referring now to FIG. 3, sensor 84 is shown in greater detail in the form of a block diagram. Sensor 84 includes a first, second and third electrodes indicated at 92, 96 and 100 respectively. Electrodes 92, 96 and 100 are for placement in contact with living tissue in order to receive electrical signals from the wearer of system 60. Electrode 96 is a ground, whereas electrodes 92 and 100 can receive varying signals in relation to ground electrode 96. Those of skill in the art will now appreciate that electrodes 92, 96 and 100 are substantially the same as prior art electrodes as found on prior art sensor 46 and generate signals accordingly.

Electrodes 92, 96 and 100 each feed into an amplifier 104 to boost the value of the signals received therefrom. In turn, amplifier 104 is connected to a filter 108 that is configured to remove any unwanted signals from signals received from electrodes 92, 96 and 100. (An example of such unwanted signals would be ambient sixty hertz signals in North America commonly found on individuals that are in the proximity of sixty hertz electrical devices.). The electrode section of the device thus detects and processes electromyographic signals at the surface (i.e. surface EMG signals). Filter 108 is a sharp analog notch filter at about sixty hertz to reduce or eliminate power line noise. Filter 108 also filters frequencies higher than about one thousand hertz. (i.e. at about a three dB cut-off at higher than about one-thousand-five-hundred Hz).

Filter 108, in turn, outputs its signal to an analog-to-digital converter 112 for converting signals from electrodes 92, 96 and 100 into digital format. Next, the signals from analog-to-digital converter 112 are outputted to an encoder 116 for placing the digitized signals into a format suitable for wireless transmission. The output from encoder 116 is then delivered to a radio 120 for transmission over link 88 via an antenna 124.

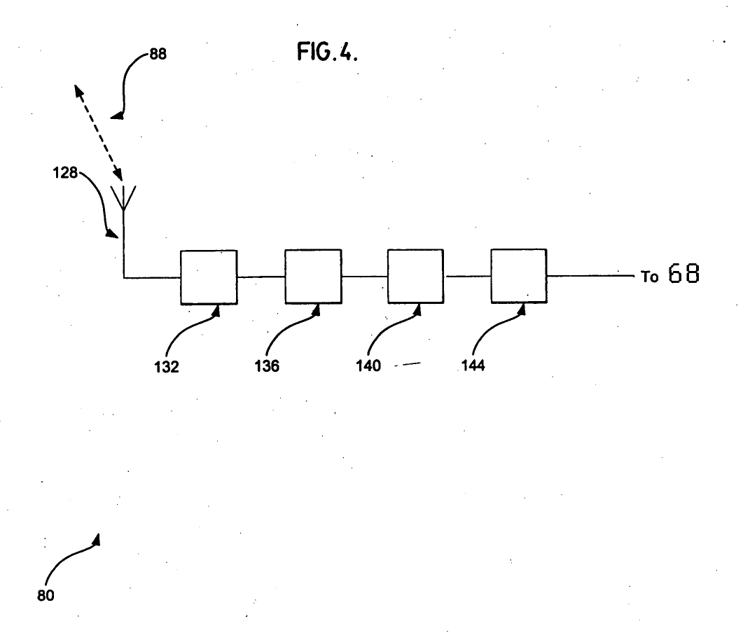

Referring now to FIG. 4, transceiver 80 is shown in greater detail in the form of a block diagram. Transceiver 80 includes its own antenna 128 which interacts with link 88. Antenna 128 is connected to a radio 132 which in turn is connected to a decoder 136. Thus, wireless signals sent from sensor 84 over link 88 are thus received at transceiver 80 and are eventually passed to decoder 136 where they are returned to substantially the same form as they arrived at encoder 116. The output from decoder 136 is then passed to a digital-to-analog converter 140, and finally to a filter 144 to remove any unwanted noise. Thus, the output from filter 144 is delivered to the controller 68 in man machine interface 76. In general, it should now be understood that the signal received at electrodes 92, 96 and 100 is delivered in a substantially readable form from the output of filter 144 using the aforementioned components. However, it is to be understood that other sets of components that transmit over a wireless link such as link 88 are within the scope of the invention.

The format of link 88 is not particularly limited. For example, frequency-Shift-Keying (“FSK”) at about 433 MHz can be used to transmit the processed signal. As another example, presently more preferred, signals are transmitted using Amplitude-Shift Keying (“ASK”) in the about 902-928 MHz Industrial Scientific and Medical (“ISM”) band. ASK modulation is used to reduce and/or minimize power consumption. If the non-digitized, raw signals are needed, they can be transmitted by changing a few components in the circuit and using Frequency Modulation (FM) transmission. EMG electrode signal channels are programmable (902-928 MHz) and because of the bandwidth of the signals and the method of transmission, transmission of multiple channels of EMG data is possible, thereby reducing the likelihood of interference from other sensors that may be nearby. The 902-928 MHz band is presently preferred in which one can operate in North America, however, there are many cordless phones and other devices that operate in this band. Therefore, to further reduce the likelihood of interference, it can be desired to include further intelligence inside the sensor 84 and transceiver 80 by assigning an ID to each sensor 84 so that transceiver 80 cannot be activated by another device.

It is also presently preferred, thought not shown in FIG. 3 for simplicity sake, to include an interface so that sensor 84 can be programmed for different frequencies (for example, 902-928 MHz), identifiers, etc. It can also be desirable that sensor 84 be programmable using software so that the output power and/or range of radio 120 is adjustable.

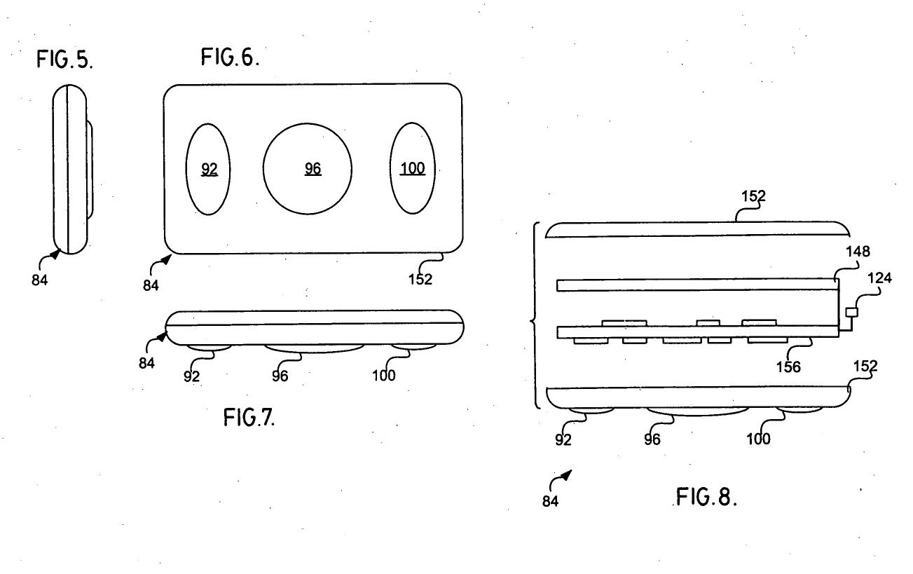

Referring now to FIGS. 5-8, various further views of sensor 84 are shown. As best seen in FIG. 8, sensor 84 includes a power supply 148 that is self contained within sensor 84. A presently preferred self-contained power supply is a single-cell rechargeable Li-Ion battery, having enough power for operating the circuits in sensor 84 for several hours of continuous operation. Also as seen in FIG. 8, sensor 84 has a two-part outer housing 152. The bottom of housing 152 frames electrodes 92, 196, 100. Housing 152 holds a printed circuit board 156 that carries the components shown in FIG. 3.

While only specific combinations of the various features and components of the present invention have been discussed herein, it will be apparent to those of skill in the art that desired subsets of the disclosed features and components and/or alternative combinations of these features and components can be utilized, as desired. For example; the electromyographic sensor described herein can be modify for use with a plurality of different types of man machine interfaces, including prosthetic limbs, computing pointing devices, etc.

The present invention provides a novel electromyographic sensor. This wireless electromyographic technology can contribute in several areas of rehabilitation, from functional electrical stimulation (“FES”) control to facial function rehabilitation (Giuffrida, 2001; Brudny, 1988; Manal, 2002). Specifically, it can be a core component of human interface devices (Barreto, 2000; Chang, 1996) for which the elimination and/or reduction of wired connections is desirable, and can improve the reliability of powered, upper-extremity prostheses by eliminating the need for wires between electrodes and control electronics. The electromyographic sensor can also enable individuals who are paralyzed to communicate with a computer or any other devices with the contraction of any muscle in the body. For individuals with conditions such as amyotrophic lateral sclerosis (“ALS”), the electromyographic sensor can allow them to use their facial muscles for Morse code generation, for example, for communication.

The electromyographic sensor can also be useful for the transmission of sensor data in gait analysis, providing EMG data which would aid in the assessment and treatment of gait anomalies (Quanbury, 1976; Letts, 1975; Winter, 1984; Perry, 1993; Harlaar, 2000). The wireless EMG system would be self-contained and smaller—an improvement over prior art systems such as the Noraxon TeleMyo 2400T. Also, the base technology of wireless data transmission could be used for transmission of other sensor data needed for gait analysis, such as shear force data. This would benefit gait analysis by enabling collection of a full data suite without tethering the subject.

As an additional example, the shape of electrodes 92, 96 and 100 can have shapes that are suitable for the location in which they are to be mounted. Thus, the shapes are not particularly limited.

The above-described embodiments of the invention are intended to be examples of the present invention and alterations and modifications may be effected thereto, by those of skill in the art, without departing from the scope of the invention which is defined solely by the claims appended hereto.

1. A self contained electromyographic sensor comprising:
electrodes for placement in contact with tissue and for receiving electrical signals therefrom;
a circuit proximally connected to said electrodes for converting said signals into a wireless transmission format; and,
a transmitter including a radio and an antenna connected to said circuit and for broadcasting said signals.

2. The sensor of claim 1 wherein said circuit is based on at least one of analog signal processing; digital signal processing; and adaptive filtering.

3. The sensor of claim 1 further comprising a receiver connected to said radio and antenna, said receiver operable to receive additional signals that include instructions for instructing how said circuit is to process said signals.

4. The sensor of claim 1 wherein said broadcasting is based on one of radio frequency, infra-red and acoustic signals.

5. The sensor of claim 1 wherein said broadcasting is based on at least one of amplitude modulated analog signals; frequency modulated analog signals; code division multiple access digital signals and orthogonal frequency multiple access digital signals.

6. The sensor of claim 1 wherein said circuit is further operable to add an identifier to said signal such that said sensor is uniquely identifiable.

7. The sensor of claim 1 wherein said transmitter includes means for varying transmission power thereof according to a desired operating range.

8. The sensor of claim 1 wherein power for said circuit is provided by a battery housed within said sensor is a battery.

9. The sensor of claim 8 wherein said battery is rechargeable via wireless means.

10. The sensor of claim 8 wherein said battery is based on NiMh or Li-ion.

11. A man machine interface based on an electromyographic sensor comprising:
electrodes for placement in contact with tissue and for receiving electrical signals therefrom;
a circuit connected to said electrodes for converting said signals into a format suitable for wireless transmission; and,
a radio and antenna connected to said circuit and for broadcasting said signals.

12. The interface of claim 11 wherein said sensor is selected from the group consisting of a pointing device; a sensor for a prosthesis; a sensor for a rehabilitation device; a sensor for a gait analysis machine.

13. A prosthetic system comprising:
an electromechanical prosthetic limb;
a controller connected to said limb for issuing movement instructions thereto, said controller including a wireless receiver;
an electromyographic sensor having electrodes for placement in contact with tissue and for receiving electrical signals therefrom; said sensor further having a circuit connected to said electrodes for converting said signals into a format suitable for wireless transmission; and, said sensor further having a transmitter connected to said circuit and for broadcasting said signals to said wireless receiver, said signal for providing input to said controller such that said controller determines corresponding movement instructions to issue to said limb.

14. A movement analysis system comprising:
a plurality of electromyographic sensors having electrodes for placement in contact with different locations of tissue, said electrodes for receiving electrical signals therefrom based on movement of said tissue; said sensors further having a circuit connected to said electrodes for converting said signals into a format suitable for wireless transmission; and, said sensor further having a transmitter connected to said circuit and for broadcasting said signals;
a computing apparatus having a receiver operable to receive said signals, said computing apparatus operable to generate a computerized representation of said movement based on said signals.

15. An electromyography method comprising the steps of:
receiving electrical signals from electrodes in contact with tissue;
converting said signals into a format suitable for wireless transmission; and,
wirelessly broadcasting said signals.
